(19) United States
(12) Patent Application Publication
(10) Pub. No.: US 2021/0252071 A1
(43) Pub. Date: Aug. 19, 2021

(54) METHOD FOR CARTILAGE REPAIR AND PRE-OSTEOARTHRITIS TREATMENT

(71) Applicant: Board of Supervisors of Louisiana State University and Agricultural and Mechanical College, Baton Rouge, LA (US)

(72) Inventor: Yufeng G. DONG, Shreveport, LA (US)

(21) Appl. No.: 16/792,171

(22) Filed: Feb. 14, 2020

(51) Int. Cl. A61K 35/28; A61K 9/00; A61K 38/17; A61P 19/02

(52) U.S. Cl. CPC A61K 35/28; A61P 19/02; A61K 38/177; A61K 9/0019

(57) ABSTRACT

Methods and compositions comprising an isolated in vitro pre-conditioned population of placenta derived mesenchymal stem cells (PMSCs), wherein the PMSCs promote de novo regeneration of cartilage when the PMSCs are administered to a subject. A method of treating one of osteoarthritis, cartilage damage or loss, and pre-osteoarthritis in a patient comprising administering to the patient a pharmaceutical composition comprising an isolated in vitro pre-conditioned population of placenta derived mesenchymal stem cells (PMSCs), wherein the in vitro pre-conditioning comprises providing a population of mesenchymal stem cells from a placenta, and culturing the population of mesenchymal stem cells in a medium comprising soluble Notch ligand Jagged1 (JAG1); and a pharmaceutically acceptable carrier. A pharmaceutical composition comprising placenta derived mesenchymal stem cells (PMSCs), Notch ligand Jagged1 (JAG1), and a pharmaceutically acceptable carrier.

### CROSS REFERENCE TO RELATED APPLICATIONS/PRIORITY

The present invention claims priority to U.S. Provisional Patent Application No. 62/805,438 filed Feb. 14, 2019, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### BACKGROUND

Osteoarthritis is a common joint disease, characterized by the degeneration of articular cartilage, that affects nearly 40 million people in the US alone. Due to the lack of blood supply, joint cartilage has limited repair potential. Therefore, using an artificial joint to replace the damaged joint has been widely practiced in clinical settings to treat patients with severe cartilage loss. Although joint replacement significantly improves the quality of life, limited activity and long-term joint loosening and revision make the new joint incomparable to the original joint. As such, disease-modifying treatments that effectively regenerate missing cartilage rather than replacing it are necessary for osteoarthritic patients.

### SUMMARY

Wherefore, it is an object of the present invention to overcome some or all of the above-mentioned shortcomings and drawbacks associated with the current technology.

Placenta-derived mesenchymal stem cells (PMSCs), a stromal cell, provide a promising cell source for tissue regeneration. However, rapid induction of PMSC chondrogenic differentiation during therapeutic transplantation remains extremely challenging. Here the inventor undertook a study to determine if Notch inhibition by soluble Jagged1 (JAG1) peptides could be utilized to accelerate PMSC-induced cartilage regeneration in a mouse post-traumatic osteoarthritis (PTOA) model. The inventor's results showed that treatment of PMSCs with soluble JAG1 significantly enhanced chondrogenesis in culture as shown by increased alcian blue staining and decreased Notch target Hes1 expression when compared to those in IgG-treated control cells. Importantly, the inventor also observed significantly enhanced cartilage formation and decreased joint inflammation when JAG1-treated PMSCs were injected into mouse PTOA knee joints. Finally, in vivo cell tracing showed that more JAG1-treated PMSCs remained in knee joint tissues and that JAG1-treated PMSCs exhibited greater PMSC chondrogenic differentiation than IgG-treated control PMSCs at 4 weeks after injection. These data indicate that transient Notch inhibition by soluble JAG1 could be used to enhance PMSC survival and chondrogenic differentiation, thereby increasing the therapeutic potential of PMSCs for cartilage regeneration.

Mesenchymal stem cells (PMSCs) derived from placental tissue are highly proliferative with multipotent differentiation capacity. Compared to the widely studied embryonic or bone marrow-derived MSCs, PMSCs have several advantages, including no ethical issues as the placenta is a medical waste generally discarded after birth, large availability, and no invasive injury for the donor. Therefore, PMSCs are an excellent cell source for regenerative medicine. Although most of the mesenchymal stem cells isolated from different sources are very similar in an undifferentiated state, differences in capacity for multilineage cell differentiation have also been observed. PMSCs have a lower potential to undergo adipogenesis, while having a higher potential to undergo chondro-osteogenesis than other MSCs. Left to be discovered was the in vivo efficacy of PMSCs for cartilage regeneration.

While PMSCs exhibit a capacity for chondrogenesis in vitro, without the stimulation of chondroinductive molecules, they are unable to spontaneously undergo chondrogenesis in vivo. To induce rapid PMSC chondrogenic differentiation, the effect of signaling pathways on stromal cell differentiation must be understood, especially the integrated signal inputs that initiate stromal cell chondrogenic differentiation. Notch signaling is a significant factor in MSC maintenance, proliferation and lineage commitment. Particularly, inhibition of Notch signaling significantly enhances limb bud-derived MSC chondrogenesis. There are four Notch receptors (Notch1-Notch4) and six Notch canonical ligands (Delta-like 1, 2, 3, 4, Jagged 1, 2) that have been identified in mice and humans. Binding of these ligands to receptors leads to proteolytic cleavage and release of the Notch intracellular domain (NICD) from the plasma membrane, which translocates to the nucleus and regulates transcription of downstream targets, most notably the Hey and Hes genes. The Notch ligand Jagged1 (JAG1) is abundantly expressed and markedly increased in cartilage from patients with osteoarthritis (OA), a disease characterized by destruction of articular cartilage due to progressive articular chondrocyte apoptosis. Additionally, activation of Notch signaling in chondrocytes induces cell death in OA mice, while inhibition of Notch signaling in chondrocytes suppresses OA development in a murine surgical model.

Although increased Notch activity is involved in OA development, its role as a therapeutic target to prevent or rescue injury-induced cartilage destruction still required clarification. Since the soluble JAG1 ligand inhibits endogenous Notch signaling in vitro, disclosed here, the inventor utilized a novel process of the negative effect of the soluble form of JAG1 peptides on Notch signaling to further enhance the PMSC therapeutic effect on cartilage repair.

Early OA, is considered a point where a patient first experiences painful symptoms and presents himself to a doctor. However, the normal joint has gone through a steady progression to become the diseased early symptomatic OA joint. It is generally agreed that OA is not a mechanical but rather a biological process. This process may well be driven by mechanics, that is, trauma or meniscectomy, but the actual breakdown of cartilage is caused by cells in the joint, predominantly the chondrocytes in the affected cartilage area. These cells start to express a different biosynthetic pattern that induces the production of metalloproteases and collagenases, which, in turn, break down the ECM, well before gross manifestation of OA disease. This impacted, pre-symptomatic condition is considered Pre-OA.

The disclosed invention is related to methods and compositions comprising an isolated in vitro pre-conditioned population of placenta derived mesenchymal stem cells (PMSCs), wherein the PMSCs promote de novo regeneration of cartilage when the PMSCs are administered to a subject. According to a further embodiment, the in vitro pre-conditioning comprises providing a population of mesenchymal stem cells from a placenta, and culturing the population of mesenchymal stem cells in a medium comprising soluble Notch ligand Jagged1 (JAG1). According to a further embodiment, the JAG1 is in a concentration of between 5.0 μg/ml and 15 μg/ml. According to a further embodiment, n the JAG1 is in a concentration of substantially 10 μg/ml. According to a further embodiment, the PMSCs are harvested from a villous tissue of the placenta.

The disclosed invention is further related to compositions and methods of treating one of osteoarthritis, cartilage damage or loss, and pre-osteoarthritis in a patient comprising administering to the patient a pharmaceutical composition comprising an isolated in vitro pre-conditioned population of placenta derived mesenchymal stem cells (PMSCs), wherein the in vitro pre-conditioning comprises providing a population of mesenchymal stem cells from a placenta, and culturing the population of mesenchymal stem cells in a medium comprising soluble Notch ligand Jagged1 (JAG1); and a pharmaceutically acceptable carrier. According to a further embodiment, the pharmaceutical composition is injected into a joint of the patient. According to a further embodiment, the joint is a knee. According to a further embodiment, the pharmaceutical composition contains between 0.1 million and 300 million PMSCs. According to a further embodiment, the pharmaceutical composition contains between 0.2 million and 30 million PMSCs. According to a further embodiment, the pharmaceutical composition contains between 0.5 million and 3 million PMSCs. According to a further embodiment, the method further comprises making no more than one injection of the pharmaceutical composition into the patient a week. According to a further embodiment, the method further comprises making one injection of the pharmaceutical composition into the patient a week for four weeks. According to a further embodiment, the pharmaceutical composition contains JAG1. According to a further embodiment, JAG1 is in a concentration of between 5.0 μg/ml and 15 μg/ml. According to a further embodiment, the JAG1 is in a concentration of substantially 10 μg/ml. According to a further embodiment, the PMSCs are harvested from a villous tissue of the placenta.

The disclosed invention is further related to methods of treatment and pharmaceutical composition comprising placenta derived mesenchymal stem cells (PMSCs), Notch ligand Jagged1 (JAG1), and a pharmaceutically acceptable carrier. According to a further embodiment, the wherein the PMSCs are harvested from a villous tissue of a human placenta. According to a further embodiment, the JAG1 is in a concentration of between 8.0 μg/ml and 12.0 μg/ml.

The presently disclosed invention relates to a stem cell based treatment that minimizes risk that will effectively regenerate missing cartilage rather than replacing it is an ideal treatment for osteoarthritic patients.

The presently disclosed invention relates to a growing Placenta-derived mesenchymal stem cells (PMSCs) into cartilage tissue

The presently disclosed invention relates to conjugating stem cells with a naturally occurring protein, JAG1, to promote cartilage formation.

The presently disclosed invention relates to optimizing cell/ligand complex injection strategy for treatment of arthritis

The presently disclosed invention relates to the use of Notch ligand and placenta stem cells for cartilage repair.

The presently disclosed invention relates to methods of preparation and injection of Notch signaling ligand treated stem cells into the site of cartilage damage or loss, to regenerate cartilage and prevent subsequent osteoarthritis

The present invention relates to pharmaceutical compositions of a therapeutic (e.g., PMSCs and JAG1) and use of these compositions for the treatment of osteoarthritis, pre-osteoarthritis, cartilage damage, and cartilage loss.

In some embodiments, the therapeutic is administered as a pharmaceutical composition that further includes a pharmaceutically acceptable excipient.

In some embodiments, the pharmaceutical composition is administered concurrently with one or more additional therapeutic agents for the treatment or prevention of osteoarthritis, pre-osteoarthritis, cartilage damage and/or cartilage loss.

In some embodiments, the therapeutic is administered as a pharmaceutical composition that further includes a pharmaceutically acceptable excipient.

The term an “effective amount” of an agent, as used herein, is that amount sufficient to effect beneficial or desired results, such as clinical results, and, as such, an “effective amount” depends upon the context in which it is being applied.

The term “pharmaceutical composition,” as used herein, includes a composition containing a compound or mixture described herein (e.g., PMSCs and JAG1), formulated with a pharmaceutically acceptable excipient and or carrier, and typically manufactured or sold with the approval of a governmental regulatory agency as part of a therapeutic regimen for the treatment of disease in a mammal.

Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, includes any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “prevent,” as used herein, includes prophylactic treatment or treatment that prevents one or more symptoms or conditions of a disease, disorder, or conditions described herein (e.g., osteoarthritis, pre-osteoarthritis, cartilage damage and/or cartilage loss.). Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

As used herein, and as well understood in the art, “treatment” includes an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also include delaying the onset of, impeding or reversing the progress of, or alleviating either the disease or condition to which the term applies, or one or more symptoms of such disease or condition.

The term “unit dosage forms” includes physically discrete units suitable as unitary dosages for human subjects and other mammals, each unit containing a predetermined quantity of active material calculated to produce the desired therapeutic effect, in association with any suitable pharmaceutical excipient or excipients.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIGS. 1A-1E are characterizations of human Placenta derived Mesenchymal Stem Cells (PMSCs). 1A-1E are representative flow cytometry histograms showing CD29, CD73, CD90 and CD166-positive PMSCs and CD34-negative PMSCs in passage 4 cultures, respectively. 1F shows quantified percentage of CD29, CD73, CD90 and CD166-positive MSCs in passage 4 cultures. Data are the means±SD of three independent experiments.

FIGS. 2A-2F shows an analysis of PMSC osteogenic and adipogenic differentiation. 2A show osteogenic differentiation that was examined by alkaline phosphatase staining and alizarin red staining without (Control) induction or with induction (Induced). Bar indicates 100 μm. FIGS. 2B and 2C show osteogenic differentiation markers Runx2 and osteocalcin were further examined by real-time PCR. FIG. 2D show adipogenic differentiation that was studied by the detection of lipid vacuoles by oil red O staining with or without induction. Bar indicates 50 μm. FIGS. 2E and 2F show adipogenic differentiation markers PPARr and C/EBPa that were inspected by real-time PCR. The data are the means±SD of three independent experiments and all the results were normalized to the internal control (*p<0.05 compared with control PMSCs without induction).

FIGS. 3A-3F shows inhibition of Notch signaling by unbound JAG1 enhances inducted PMSC chondrogenic differentiation. 3A shows quantification of gene expression of the Notch target gene Hes1 indicates a significant increase (p<0.05) in cells with surface-bound (coated) JAG1 (10 μg/ml) treatment, and a reduced expression was observed in cells treated with a single treatment of unbound (Floating) JAG1 (10 μg/ml) for up to 5 days. 3B are luciferase assays that showed a significant decrease in Notch-responsive reporter activity in bound JAG1-treated MSCs and reduced activity in day 1 and day 3 with unbound JAG1 treatment. The data are the means±SD of three independent experiments performed in duplicate, and the gene expression level in control cells (Co) was set at 1 (*p<0.05 compared with control). 3C shows an increase in chondrogenesis was observed in unbound JAG1-treated PMSCs at day 14, with stronger staining of alcian blue and type II collagen (Col-II). Scale bars, 100 μm. 3D-3F show quantification of gene expression of cartilage matrix aggrecan and Col-II, as well as transcription factor Sox9, using RNA from day 14 culture pellets. Data are the means±SD of three independent experiments performed in triplicate, and all the results were normalized to internal control (*p<0.05 compared with control PMSCs (Co).

FIGS. 4A-4C show mice develop more severe osteoarthritis without PMSC injection. At week 4 post-intra-articular injection of PBS, JAG1 (10 μg/ml) in PBS, PMSCs with IgG (10 μg/ml) or JAG1 (10 μg/ml), right knee joints were harvested for histological assessment. 4A are representative images of medial compartment of knee sections from MLI mice with injection of PBS (Control), JAG1, PMSC+IgG and PMSC+JAG1 stained with alcian blue/orange G. 4B shows osteoarthritic changes in knee joints (n=12) as quantified with Osteoarthritis Research Society International (OARSI) score. Data are the means±SD (*p<0.05 compared with control; #P<0.05 between two groups). 4C is immunohistochemistry staining (IHC) of chondrogenic marker type II collagen (Col-II) and type X collagen (Col-X) in tibia articular cartilage from 8-week MLI mice with or without PMSC injection. Increased Col-X expression was detected, and more Col-X-positive cells were located toward the articular surface in control and PMSC+IgG mice, not in PMSC+JAG1 mice 8-weeks after MLI surgery (Col-X positive cells: red arrowheads)

FIG. 5A-5D show PMSCs in the mouse knee joint. In 5A, Qtracker® reagent-labeled PMSCs showed strong red fluorescence after 1 h of incubation in the culture. In 5B, mouse knee joints injected with 0.2 million red fluorescent-labeled JAG1-treated PMSCs showed more live PMSCs invading synovial tissue surrounding the artificial cartilage than joints injected with untreated PMSCs at 4 weeks after cell injection. 5C shows immunohistochemistry staining (IHC) of human PMSCs using the anti-human nuclear antigen antibody in tibia articular cartilage from 8-week MLI mice (human nuclear antigen positive cells: red arrowheads). 5D shows H&E staining of subchondral bone in the mouse knee joint from control, PMSC and JAG1/PMSC groups at 4 weeks after cell injection.

FIGS. 6A-6D show a treatment inhibits chondrocyte apoptosis and inflammation in the mouse OA joint. FIG. 6A Top panel: The representative images of TUNEL staining of the tibia plateau from a control OA mouse injected with 8 μl PBS. Middle panel: The stained tibia plateau from a control OA mouse injected with 8 μl PBS containing 0.2 million PMSCs and IgG (10 μg/ml). Low panel: The stained tibia plateau from a control OA mouse injected with 8 μl PBS containing 0.2 million PMSCs and JAG1 (10 μg/ml). FIG. 6B Quantification of TUNEL-positive cells in areas of the stained tibia plateau (n=12). Data are the means±SD (*p<0.05 compared with control; #P<0.05 between two groups).

FIG. 6C Relative mRNA expression of proinflammation cytokines was analyzed by quantitative real-time PCR after synovial tissue obtained from OA mice was treated with PMSCs with or without JAG1 for 4 weeks. The data are the means±SD (*p<0.05 compared with control). FIG. 6D Relative mRNA expression of the Notch target gene Hes1 in synovial tissue was analyzed by quantitative real-time PCR. Data are the means±SD (*p<0.05 compared with control; #P<0.05 between two groups)

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

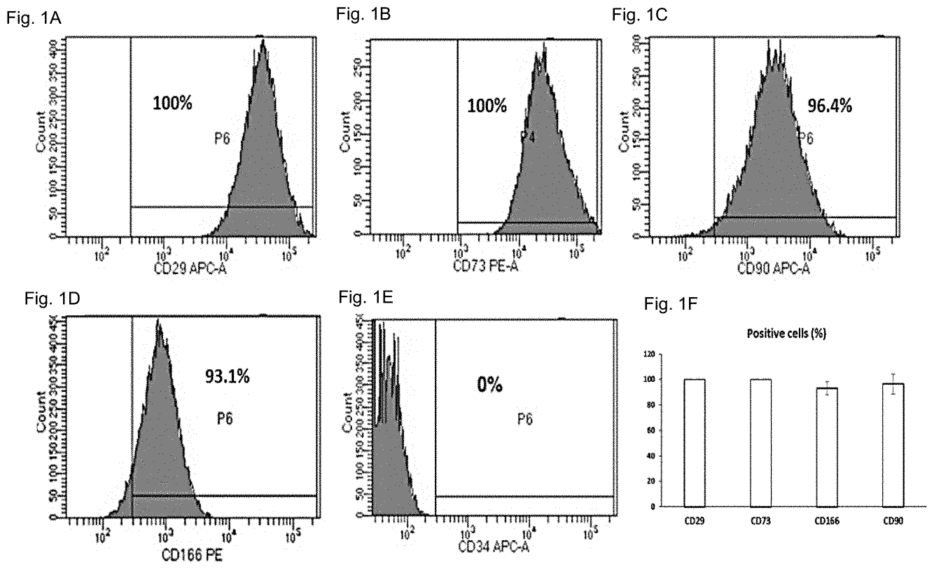

Turning now to FIGS. 1A-6D, a brief description concerning the various components of the present invention will now be briefly discussed.

The isolation of PMSCs was performed following a procedure based on the adherence of cells to the surface of culture dishes. After 4 passages for expansion, most PMSCs showed a fibroblast-like morphology. Flow cytometry data indicated that PMSCs was positive for stem cell surface markers CD29, CD73, CD90, and CD166 (FIGS. 1A, 1B, 1C, 1D), and negative for endothelial marker CD34 (FIG. 1E). The final quantification of these positive cell populations was further shown in FIG. 1F.

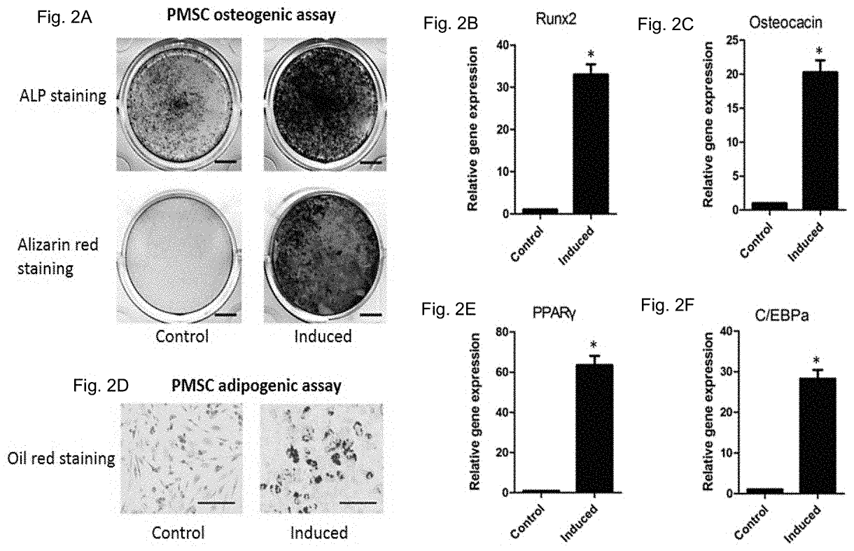

Results from in vitro differentiation assays clearly showed that these PMSCs could be successfully induced into osteoblastic cells by showing enhanced ALP and Alizarin red staining (FIG. 2A) when cultured in osteogenic medium. PCR data confirmed that enhanced osteogenesis by showing increased expression of Runx2 (FIG. 2B) and osteocalcin (OC) (FIG. 2C), markers of osteoblasts. For adipogenic differentiation, intracellular lipid accumulation was noted in cells cultured with adipogenic medium (FIG. 2D), indicating that PMSC could be differentiated into adipocytes in cultures. As expected, expression of adipogenic marker genes PPARr (FIG. 2E) and C/EBPa (FIG. 2F) were markedly increased.

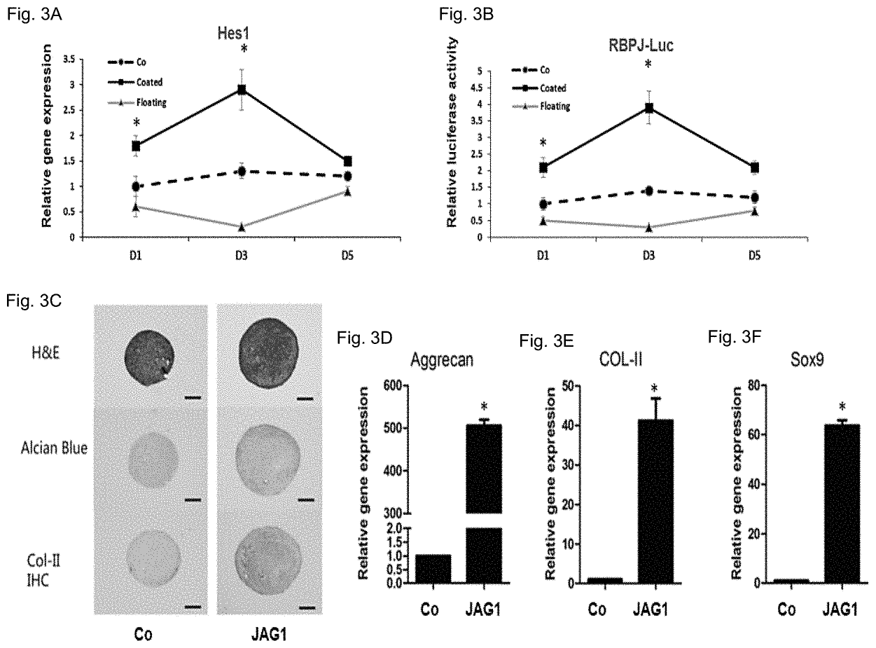

To verify the different effect of surface bounded and unbounded JAG1 on Notch signaling in PMSCs, the inventor cultured cells in either JAG1-coated plates or plates containing soluble JAG1 in medium for up to 5 days. Surface-bounded JAG1 significantly induced activity of Notch signaling. In contrast, addition of JAG1 in medium resulted in a reduced Notch activity in cells (FIG. 3A). Notch signaling responsive reporter RBPJ-Luc activity in PMSCs was also significantly enhanced by bounded JAG1 and reduced by unbounded JAG1 (FIG. 3B) confirming that unbounded soluble JAG1 is capable to inhibit Notch signaling in PMSCs. More importantly, treatment with soluble unbounded JAG1 formed a larger pellet and induced a stronger Alcian blue staining when compared to control cells treated with IgG peptides. Enhanced Col-II immune-staining further demonstrated that more cartilage matrix synthesized in JAG1-treated pellets (FIG. 3C). RT-PCR data showed a significantly enhanced chondrogenic markers aggrecan (FIG. 3D) and Col-II (FIG. 3E) gene expression when compared to control cells. Finally, a significantly increased expression of Sox9, one critical chondrogenic inducer, was observed in JAG1-treated cell pellets (FIG. 3F). Together these data suggest that inhibition of Notch signaling by unbounded JAG1 ligand further promotes PMSC chondrogenic differentiation.

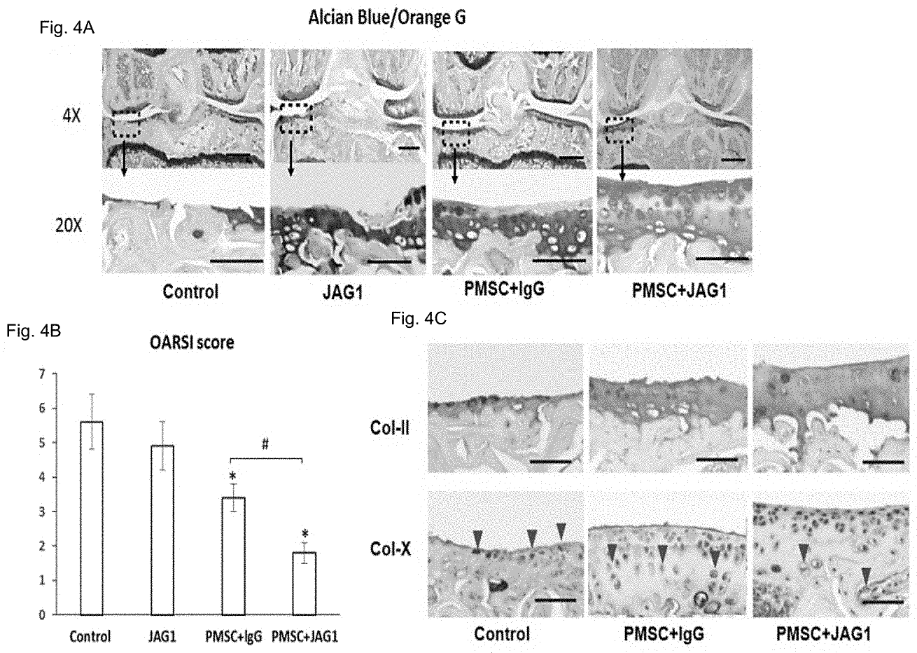

To study PMSC-mediated cartilage regeneration, the intra-knee joint injection of JAG1/PMSCs or IgG-treated PMSCs (IgG/PMSCs) was performed using OA mouse model. The inventor's histology staining (FIG. 4A) showed that severe OA-like defects were indeed developed in control mice at 8 weeks in PBS-injected group. JAG1 alone injection group also showed a severe loss of artificial cartilage with only small Alcian blue-positive area observed. In contrast, more Alcian blue-positive cartilage was observed in IgG/PMSC injected groups when compared with PBS or JAG1 alone treated groups. More importantly, JAG1/PMSCs induced a thicker cartilage formation compared to IgG/PMSCs by showing a wider Alcian blue positive top layer of cartilage. Consistent with histological observation, the evaluation using OARSI scoring system (the higher the score, the greater the articular cartilage degeneration) revealed that although there was no significant difference between PBS and JAG1 alone groups, a significant reduction of cartilage degeneration was observed in mice with JAG1/PMSC injection when compared with IgG/PMSC groups (FIG. 4B). Our IHC data showed an increased expression level of Col-II in the cartilage from JAG1/PMSC group when compared with IgG/PMSC group (FIG. 4C), indicating that JAG1 induces more cartilage matrix formation when combined with PMSCs. Interestingly, a strong expression of type X collagen (a marker of chondrocyte hypertrophic transformation) was observed in the top layer of cartilage with injection of IgG/PMSCs. However, in mice with injection of JAG1/PMSCs, the Col-X expression was observed only in the deep zone of articular cartilage (FIG. 4C).

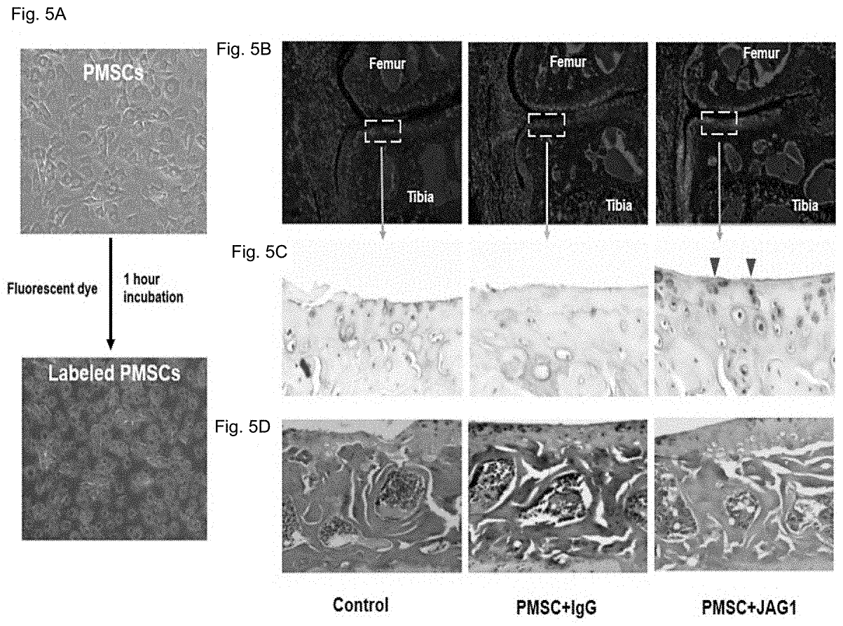

To track PMSCs in vivo engraft and survive, cells were first labeled by red fluorescent dye that can be traced through several generations. After one-hour incubation, the cell labeling image showed that 99% of PMSCs were successfully labeled by red dye without any noticeable cell death (FIG. 5A). More importantly, knee joint tissues from the mice with JAG1/PMSC injection showed more red fluorescent signals remained in the surgery joints at 4 weeks after injection when compared with the IgG/PMSCs injected surgery joints suggest that more live cells in JAG1/PMSC injected groups since the dead cells do not show red fluorescent (FIG. 5B). Interestingly, these labeled live PMSCs were only observed at the space between tibia plateau and synovial membrane, not on the cartilage surface or inside of artificial cartilage. Since fluorescent in labeled PMSCs will also lost signals after extensive proliferation and cell differentiation, we further traced differentiated PMSCs using anti-human nuclear antigen antibody. Our IHC data clearly showed no positive cells were observed on the surface of cartilage in IgG/PMSC injected mouse. However, a number of positive cells were found on the top layer of artificial cartilage in mouse with injection of JAG1/PMSCs (FIG. 5C). Finally, the inventor's H&E staining of tibia plateau showed no significant difference was observed regarding the structure and density of subchondral bone among mice from three different treatment groups (FIG. 5D) suggesting neither PMSCs nor JAG1 has effect on deeper layer subchondral bone when short-term treatments are used.

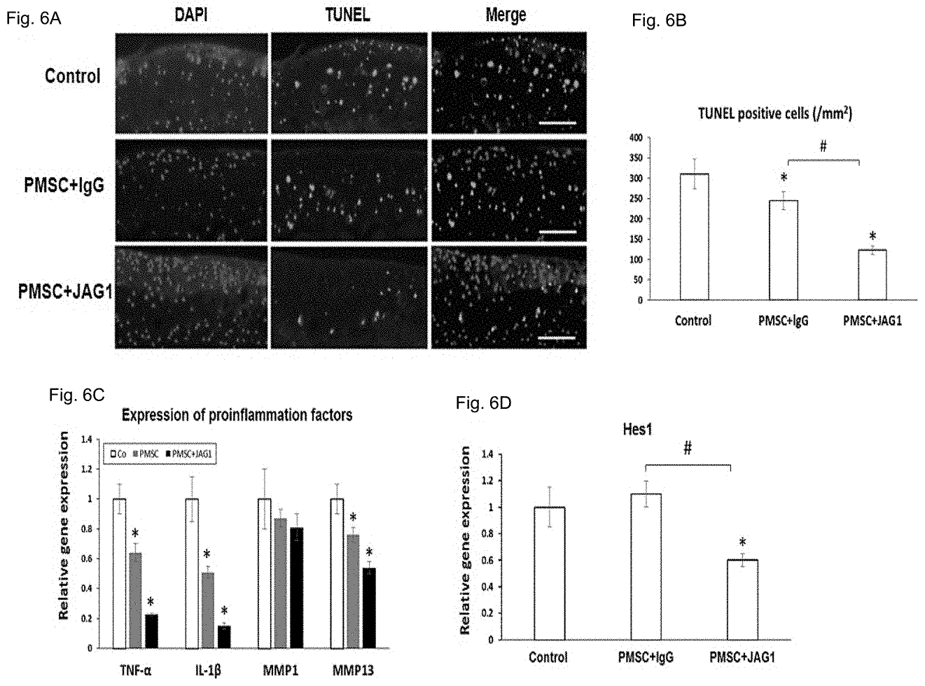

The inventor next investigated the possible role of PMSCs in modulating the apoptosis in OA articular cartilage. The inventor's TUNEL staining data clearly showed that IgG/PMSC injection significantly reduced chondrocyte apoptosis that was induced by MLI surgery in articular cartilage (FIG. 6A). More importantly, injection of JAG1/PMSCs led to even more reduction of chondrocyte apoptosis when compared with IgG/PMSCs (FIG. 6B). We further measured the expression of proinflammatory factors using RNA extracted from synovial membrane surrounding OA knee joint. Our PCR data showed that while no significant change was noticed in expression of MMP1, a protein involved in the breakdown of extracellular matrix in normal physiological processes, inhibition of TNF-α, IL-1β, and MMP13 was indeed observed with PMSC injection, and this inhibition was further potentiated by adding JAG1 treatment (FIG. 6C). In addition, Notch target gene Hes1 expression in synovial tissue was also significantly reduced by treatments with JAG1 suggesting decreased Notch activity in synovial tissues (FIG. 6D).

To rapidly regenerate cartilage, one exciting strategy involves the use of the multi-potent MSCs. Since MSCs home to and are preferentially attracted to diseased tissue rather than to intact tissue, intra-articular injection of MSCs may be used to deliver cells to joints. Allogeneic MSCs may be used as these cells possess low immunogenicity. In this study, MSCs isolated from human placentas were first characterized for multipotency. The inventor's in vitro differentiation assays clearly showed that these cells could be differentiated into osteoblasts, chondrocytes, and adipocytes that makes PMSCs an ideal cell source for the tissue engineering and regenerative medicine. To further induce PMSC rapid chondrogenic differentiation, the inventor utilized Notch signaling ligand to promote MSC differentiation for accelerated chondrogenesis. The inventor's joint injection strategy not only induces several transient Notch inhibitions which promote injected PMSC to initiate chondrogenic differentiation, but also avoids possible side effects caused by constitutive Notch inhibition. More importantly, the anti-inflammation and anti-apoptotic effect of PMSC on arthritis joint could be further enhanced by inhibition of Notch signaling via JAG1 treatment.

Materials and Methods

Human PMSC isolation and culture: Placentas delivered by normal pregnant women were collected immediately after delivery. Since the placenta is considered a medical waste, no consent from the patients was needed. Collection of human placentas for MSC isolation was approved by the IRB at Louisiana State University Health Science Center—Shreveport (LSUHSC-S), and MSC isolation was processed at the Department of Gynecology and Obstetrics, LSUHSC-S. The procedures for PMSC isolation and culture were performed. Briefly, villous placenta tissue was separated by sterile dissection from different cotyledons, excluding chorionic and basal plates. After extensive washing with ice-cold phosphate-buffered saline (PBS), villous tissue was digested with trypsin (0.125% trypsin solution containing 0.1 mg/ml DNase I and 5 mM MgCl2) in Dulbecco's Modified Eagle's Medium (DMEM) at 37° C. for 90 min. Digested cells were collected and cultured in DMEM supplemented with 10% fetal bovine serum (FBS). PMSCs started to grow in 3-5 days. At ˜80% confluence, the cells were passaged with TrypLE™ Express (Invitrogen, Carlsbad, Calif., USA). Passage 4 (P4) PMSCs were characterized by flow cytometry using the following antibodies: CD29-APC, CD73-PE, CD90-APC, and CD166-APC (Abcam, Cambridge, Mass., USA). CD34-APC served as a negative control. The nontransmembrane, soluble forms of the JAG1 peptides encoding the Delta/Serrate/lag-2 domain (DSL) (CDDYYYGFGCNKFCRPR) (AnaSpec Inc., USA) were used to inhibit Notch signaling in PMSCs, and IgG peptides were used as the control.

Luciferase assay: JAG1 peptide-coated plates were prepared, and PMSCs cultured in regular or JAG1-coated plates were then transfected with Notch-responsive RBPJ-Luc and SV40-Renilla-Luc in the presence of Lipofectamine 2000 (Invitrogen). Forty-eight hours after transfection and treatment, lysates were analyzed with a Dual Luciferase Assay Kit (Promega).

Osteogenic and adipogenic differentiation: To induce PMSC osteogenic differentiation, P4 PMSCs were cultured with DMEM supplemented with 10% FBS, ascorbic acid (50 mg/ml), 1 μmol/L dexamethasone, and 3-glycerophosphate (10 mM) for up to 14 days. Alkaline phosphatase (ALP) and alizarin red staining were performed to visualize osteogenic differentiation. RNA was also extracted for osteogenic gene expression of Runx2 and Osteocalcin. Adipogenic differentiation was induced by medium containing insulin (10 mg/ml), dexamethasone (1 mM), and 3-isobutyl-1-methylxanthine (0.5 mM) for up to 21 days. Oil Red O staining, PPARγ and C/EBPa gene expression were used to monitor adipogenic differentiation.

Chondrogenic cell pellet culture: PMSC pellets were obtained by centrifuging 2.5×10⁴ cells at 250×g for 5 min in 15 ml polypropylene conical tubes. Pellets were cultured for 14 days in 0.5 ml of serum-free MesenCult™-Chondrogenic differentiation medium (Stem cell Technologies, Vancouver, Canada) containing soluble JAG1 (10 μg/ml) or IgG (10 μg/ml) peptides. Harvested pellets were fixed in 4% paraformaldehyde (PFA), dehydrated, and embedded in paraffin. Serial sections of 5 μm were stained with alcian blue/orange G. Immunohistochemical (IHC) analysis for Col-II was performed.

In vivo experiment: All mouse experiments were performed according to the protocol approved by the Animal Care and Use Committee of the Louisiana State University Health Sciences Center. The mouse experimental OA model was induced in the right knee joint by meniscal/ligamentous injury (MLI). 36 C57BL6/J mice were anesthetized and shaved on right knee joints for aseptic surgery. The medial joint capsule adjunct to the patellar tendon was incised with a blade to expose the medial meniscotibial ligament and the entire medial meniscus. After carefully cutting off the medial meniscus and washing with saline to remove tissue debris, the medial capsular incision was then closed with sutures. For 4 weeks after MLI surgery, 8 μl PBS solution containing 0.2 million PMSCs mixed with JAG1 (10 μg/ml) or IgG (10 μg/ml) were injected (once a week for 4 weeks) into the joint cavity from the medial edge of the patellar ligament in each group (n=12). To track the in vivo contribution of cells, PMSCs were labeled by red fluorescent dye using the Qtracker® 585 Cell Labeling Kit (Thermo Fisher Scientific). Mice were euthanized 4 weeks after intra-articular injection (8 weeks after MLI surgery).

Knee joint samples for histology were fixed in 4% formaldehyde, decalcified in 10% ethylene diamine-tetra acetic acid (EDTA) solution, and embedded in paraffin. Tissue sections were observed using a fluorescence microscope for labeled PMSCs and stained with H&E and alcian blue/orange G for histological scoring. In a blinded fashion, three examiners used a modified Osteoarthritis Research Society International (OARSI) score to evaluate the histological condition of the articular cartilage surface as described before. In brief, each section was assigned a grade 0-6: 0, normal cartilage; 0.5, loss of alcian blue staining without structural changes; 1, small fibrillations without loss of cartilage; 2, vertical clefts down to the layer below the superficial layer; 3-6, vertical clefts or erosion to the calcified cartilage [<25% (grade 3), 25-50% (grade 4), 50-75% (grade 5) and >75% (grade 6) of the articular surface is affected]. The maximal score was used to represent severity of the OA progression for each mouse. Immunohistochemical (IHC) analysis for Col-II and Col-X was performed. IHC for tracing human cells in knee joint tissue was also performed using anti-human nuclear antigen antibody (ab191181, Abcam, USA). Chondrocyte apoptosis in tibia articular cartilage was further determined by the Terminal deoxynucleotidyl transferase (TdT) dUTP Nick-End Labeling (TUNEL) assay using an in-situ Cell Death Detection Kit (Roche Diagnostics, Mannheim, Germany). For synovial tissue RNA extraction, synovial membrane surrounding the knee joint was collected and homogenized in cold Trizol reagent (Life Technologies). Chloroform was mixed with the lysate and, following centrifugation, the aqueous RNA layer was transferred to a new microcentrifuge tube. Prechilled isopropanol was then mixed with the RNA layer. Following centrifugation, the RNA pellet was washed with 70% ethanol and redissolved in RNase-free water. The concentration/purity of the RNA sample(s) was measured using the NanoDrop 2000.

Quantitative RT-PCR: Total RNA isolated from PMSCs, and synovial tissues was reverse transcribed to cDNA using an iScript cDNA Synthesis Kit (Bio-Rad, Hercules, Calif., USA) according to the manufacturer's instructions. 3-actin was used as an internal control. For gene expressions, RT-PCRs were performed on ABI 7900HT fast Real-Time PCR System using primers for Notch target Hes1, chondrogenic (Col-II, Aggrecan and SOX9), osteogenic (Runx2 and Osteocalcin), adipogenic (PPARr and C/EBPa), and proinflammation (TNF-α, IL-1β, MMP-1, MMP-13) marker genes.

Statistical analysis: The above experiments were repeated at least three times independently. All data are presented as the mean±SD. Statistical significance among the groups was assessed using one-way analysis of variance (ANOVA). The level of significance was P<0.05.

Result

Characterization of PMSCs: The isolation of PMSCs was performed following a procedure based on the adherence of cells to the surface of culture dishes. After 4 passages for expansion, most PMSCs showed a fibroblast-like morphology. Flow cytometry data indicated that the PMSCs were positive for stromal cell surface markers CD29, CD73, CD90, and CD166 (FIGS. 1A-1D), and negative for endothelial marker CD34 (FIG. 1E). The final quantification of these positive cell populations was further shown in FIG. 1F. More importantly, the results from in vitro differentiation assays clearly showed that these PMSCs could be successfully induced into osteoblastic cells as indicated by the enhanced ALP and alizarin red staining (FIG. 2A) when cultured in osteogenic medium for 14 days. PCR data confirmed enhanced osteogenesis by showing increased expression of Runx2 (FIG. 2B) and osteocalcin (OC) (FIG. 2C), which are both markers of osteoblasts. For adipogenic differentiation, intracellular lipid accumulation was noted in cells cultured with adipogenic medium (FIG. 2D), indicating that PMSCs could be differentiated into adipocytes in cultures. Expression of adipogenic marker genes PPARr (FIG. 2E) and C/EBPa (FIG. 2F) were markedly increased in culture with adipogenic medium.

Enhanced chondrogenic differentiation in PMSC culture with JAG1 treatment: To further investigate the effects of JAG1 on chondrogenic potential in PMSCs, a cell pellet culture assay was performed. Soluble extracellular forms of JAG1 DSL ligands can function to regulate Notch signal transduction either positively or negatively, depending on the context. Surface-bound JAG1 activates Notch signaling in vitro, while, in contrast, floating ligands in medium inhibit Notch signaling by competing with membrane-bounded DSL ligands for Notch receptors. To verify the different effects of surface-bound and unbound JAG1 on Notch signaling in PMSCs, the inventor cultured cells in either JAG1-coated plates or plates containing soluble JAG1 in the medium for up to 5 days and then monitored the Notch activation by PCR and luciferase assay. Since the inventor's data showed that surface-bound JAG1 at 10 μg/ml can more effectively activate Notch signaling in MSCs than concentrations at 5 μg/ml and 15 μg/ml, the inventor decided to use 10 μg/ml JAG1 to treat cells in the inventor's subsequent experiments. Surface-bound JAG1 significantly induced expression of the Notch target Hes1 at day 1 in culture, and the expression peak was observed at day 3, followed by a decrease at day 5. In contrast, the addition of JAG1 in the medium resulted in a reduced expression of Hes1 at day 1 and a greater reduction at day 3 (FIG. 3A). Similar to the PCR data, the Notch signaling-responsive reporter (RBPJ-Luc) activity in PMSCs was also significantly enhanced by bound JAG1 and reduced by unbound JAG1 (FIG. 3B) for up to 5 days after a single treatment, confirming that unbound soluble JAG1 is capable of inhibiting Notch signaling in PMSCs.

To further explore the effect of unbound JAG1 on PMSC chondrogenic differentiation, the inventor performed histological and immuno-staining on pellet cultures treated with chondrogenic medium containing either IgG or JAG1 for 14 days. Interestingly, treatment with soluble unbound JAG1 formed a larger pellet and induced stronger alcian blue staining than treatment with IgG peptides. Enhanced Col-II immune-staining further demonstrated that more cartilage matrix was synthesized in the JAG1-treated pellets than IgG-treated pellets (FIG. 3C). RT-PCR data showed significantly enhanced gene expression of chondrogenic markers, aggrecan (FIG. 3D) and Col-II (FIG. 3E) compared to the expression in control cells. Finally, a significantly increased expression of Sox9, a critical chondrogenic inducer, was observed in the JAG1-treated cell pellets compared to that in the IgG-treated cell pellets (FIG. SF). Together, these data evidence that inhibition of Notch signaling by the unbound JAG1 ligand further promotes the PMSC chondrogenic differentiation induced by specific chondrogenic medium.

JAG1-treated PMSCs induce cartilage repair in experimental OA: Having identified a clear induction of PMSC chondrogenesis by unbound JAG1, the inventor next tested whether JAG1-treated PMSCs (JAG/PMSCs) could be utilized to enhance cartilage regeneration in a mouse OA model. To create an OA mouse model, MLI surgery was performed in 12-week-old mice. In this-modified experimental OA model, an OA-like phenotype in knee joints was rapidly induced as early as 4 weeks after surgery indicated by fibrillation, clefting and cartilage degradation since a portion of the meniscus was also removed.

To study PMSC-mediated cartilage regeneration, intraknee joint injection of JAG1/PMSCs or IgG-treated PMSCs (IgG/PMSCs) was performed at 4 weeks after MLI surgery when the OA phenotype was established. To reduce the injection-stimulated joint swelling, the inventor injected only a small amount of PMSCs (0.2 million) into OA joints starting at 4 weeks after MLI surgery. After four injections (once a week) in 4 weeks, the knee joints were then harvested for analysis. Consistent with previous findings, the inventor's histology staining (FIG. 4A) showed that severe OA-like defects were indeed developed in control mice at 8 weeks after MLI surgery in the PBS-injected group. Similar to the PBS control group, the JAG1 alone injection group also showed a severe loss of artificial cartilage with only a small alcian blue-positive area observed. In contrast, more alcian blue-positive cartilage was observed in the IgG/PMSC-injected groups than in the PBS or JAG1 alone-treated groups. More importantly, JAG1/PMSCs induced thicker cartilage formation than IgG/PMSCs, as shown by a wider alcian blue-positive top layer of cartilage. Consistent with histological observations, the evaluation using the OARSI scoring system (the higher the score, the greater the articular cartilage degeneration) revealed that although there was no significant difference between the PBS and JAG1 alone groups, a significant reduction in cartilage degeneration was observed in mice with JAG1/PMSC injection when compared to that in the IgG/PMSC groups (FIG. 4B). Since no significant difference was observed in OARSI scores between the PBS and JAG1 alone injection groups in MLI mice, in subsequent experiments, only PBS control, IgG/PMSC and JAG1/PMSC groups were used to identify possible mechanisms.

The inventor's IHC data showed an increased expression of Col-II in the cartilage from the JAG1/PMSC group compared to that in the IgG/PMSC group (FIG. 4C), indicating that JAG1 induces more cartilage matrix formation when combined with PMSCs. Interestingly, a strong expression of type X collagen (a marker of chondrocyte hypertrophic transformation) was observed in the top layer of cartilage with injection of IgG/PMSCs. However, in mice with injection of JAG1/PMSCs, Col-X expression was only observed in the deep zone of articular cartilage (FIG. 4C), suggesting that while PMSC alone enhances cartilage formation, it fails to prevent chondrocyte terminal differentiation. In the control mice, since the superficial and mid zone of the cartilage was no longer present at 8 weeks after MLI surgery, Col-X expression at this time point was observed only in the thin surface layer, which is the exposed deep zone of the original articular cartilage.

JAG1 treatment enhances PMSC survival and chondrogenic differentiation in vivo: To track the in vivo engraftment and survival of PMSCs, cells were first labeled with red fluorescent dye that can be traced through several generations. After a one-hour incubation, cell labeling showed that 99% of PMSCs were successfully labeled with red dye without any noticeable cell death (FIG. 5A). More importantly, knee joint tissues from the mice with JAG1/PMSC injection had more red fluorescent signals remaining in the surgery joints 4 weeks after injection than the IgG/PMSC-injected surgery joints, suggesting that there were more live cells in the JAG1/PMSC-injected groups since the dead cells do not show red fluorescence (FIG. 5B). Interestingly, these labeled live PMSCs were only observed in the space between the tibia plateau and synovial membrane, not on the cartilage surface or inside of the artificial cartilage. Since fluorescent-labeled PMSCs also lose signal after extensive proliferation and cell differentiation, the inventor further traced differentiated PMSCs using the ant-human nuclear antigen antibody. The inventor's IHC data clearly showed that no positive cells were observed on the surface of cartilage in IgG/PMSC-injected mice. However, a number of positive cells were found on the top layer of the artificial cartilage in mice given injection of JAG1/PMSCs (FIG. 5C). Finally, the inventor's H&E staining of the tibia plateau showed no significant difference in the structure or density of subchondral bone among mice from the three different treatment groups (FIG. 5D), suggesting that neither PMSCs nor JAG1 had an effect on deeper-layer subchondral bone with short-term treatments.

JAG1-treated PMSCs inhibit chondrocyte apoptosis and inflammation in mouse OA joint: Since the pathogenic mechanism of OA involves accelerated chondrocyte apoptosis, and the extent of chondrocyte apoptosis is correlated with severity of OA, the inventor next investigated the possible role of PMSCs in modulating apoptosis in OA articular cartilage. The inventor's TUNEL staining data clearly showed that IgG/PMSC injection significantly reduced the chondrocyte apoptosis that was induced by MLI surgery in articular cartilage (FIG. 6A). More importantly, injection of JAG/PMSCs led to an even greater reduction in chondrocyte apoptosis than injection of IgG/PMSCs (FIG. 6B). Since synovial tissue-produced proinflammatory cytokines, such as TNF-α, IL-1β, matrix metalloproteinase 1 (MMP1) and matrix metalloproteinase 13 (MMP13), have been shown to initiate local inflammatory responses and lead to the progression of cartilage cell apoptosis in the pathogenesis of OA, the inventor further measured the expression of these proinflammatory factors using RNA extracted from the synovial membrane surrounding the OA knee joint. The inventor's PCR data showed that while no significant change was noticed in the expression of MMP1, a protein involved in the breakdown of extracellular matrix in normal physiological processes, inhibition of TNF-α, IL-1β, and MMP13 was observed with PMSC injection, and this inhibition was further potentiated by the addition of JAG1 (FIG. GC). In addition, the expression of the Notch target gene Hes1 in synovial tissue was also significantly reduced by treatments with JAG1, suggesting decreased Notch activity in synovial tissues (FIG. 6D).

Pharmaceutical Compositions

The methods described herein can also include the administrations of pharmaceutically acceptable compositions that include the therapeutic. When employed as pharmaceuticals, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention can be administered alone, or in a mixture, in the presence of a pharmaceutically acceptable excipient or carrier. The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 22^{nd} Ed., Gennaro, Ed., Lippencott Williams & Wilkins (2012), a well-known reference text in this field, and in the USP/NF (United States Pharmacopeia and the National Formulary), each of which is incorporated by reference. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients are described in Handbook of Pharmaceutical Excipients, 8^{th} Edition, Sheskey et al., Eds., Pharmaceutical Press (2017), which is incorporated by reference.

The methods described herein can include the administration of a therapeutic, or other therapeutic agents.

The pharmaceutical compositions can be formulated so as to provide immediate, extended, or delayed release of the active ingredient after administration to the patient by employing procedures known in the art.

The compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or, from about 100 mg to about 250 mg of the active ingredient. For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

Parenteral Administration

Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

Dosing Regimes

The present methods for treating osteoarthritis, pre-osteoarthritis, cartilage damage and/or cartilage loss are carried out by administering a therapeutic for a time and in an amount sufficient to result in decreased cartilage damage or increase healthy cartilage tissue.

The amount and frequency of administration of the compositions can vary depending on, for example, what is being administered, the state of the patient, and the manner of administration. In therapeutic applications, compositions can be administered to a patient suffering from osteoarthritis, pre-osteoarthritis, cartilage damage and/or cartilage loss in an amount sufficient to relieve or least partially relieve the symptoms of the osteoarthritis, pre-osteoarthritis, cartilage damage and/or cartilage loss and its complications. The dosage is likely to depend on such variables as the type and extent of progression of the osteoarthritis, pre-osteoarthritis, cartilage damage and/or cartilage loss, the severity of the osteoarthritis, pre-osteoarthritis, cartilage damage and/or cartilage loss, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of the osteoarthritis, pre-osteoarthritis, cartilage damage and/or cartilage loss or slowing its progression.

The amount of therapeutic per dose can vary. For example, a subject can receive from about 0.1 μg/kg to about 10,000 μg/kg. Generally, the therapeutic is administered in an amount such that the peak plasma concentration ranges from 150 nM-250 μM.

Exemplary dosage amounts can fall between 0.1-5000 μg/kg, 100-1500 μg/kg, 100-350 μg/kg, 340-750 μg/kg, or 750-1000 μg/kg. Exemplary dosages can 0.25, 0.5, 0.75, 10, or 2 mg/kg. In another embodiment, the administered dosage can range from 0.05-5 mmol of therapeutic (e.g., 0.089-3.9 mmol) or 0.1-50 μmol of therapeutic (e.g., 0.1-25 μmol or 0.4-20 μmol).

The frequency of treatment may also vary. The subject can be treated one or more times per day with therapeutic (e.g., once, twice, three, four or more times) or every so-many hours (e.g., about every 2, 4, 6, 8, 12, or 24 hours). Preferably, the pharmaceutical composition is administered 1 or 2 times per 24 hours. The time course of treatment may be of varying duration, e.g., for two, three, four, five, six, seven, eight, nine, ten or more days. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

The in JAG1 vitro pre-conditioned PMSCs may be administered to a subject in need thereof in an amount effective to treat the osteoarthritis, pre-osteoarthritis, cartilage damage and/or cartilage loss, which can be readily determined by an ordinary artisan. Further, the in vitro pre-conditioned PMSCs may be administered by any method known in the art. For example, the in vitro pre-conditioned PMSCs can be administered by injection into a target site of a subject, typically via a delivery device, such as a tube, e.g., catheter. More typically, the tube additionally contains a needle, e.g., a syringe, through which the cells can be introduced into the subject at a desired location. Specific, non-limiting examples of administering cells to subjects may also include administration by subcutaneous injection, intramuscular injection, or intravenous injection. If administration is intravenous, an injectable liquid suspension of cells can be prepared and administered by a continuous drip or as a bolus. Cells may also be inserted into a delivery device, e.g., a syringe, in different forms. For example, the cells can be suspended in a pharmaceutically acceptable carrier as described above contained in the delivery device.

Typically, the cells are administered locally (for example by direct application under visualization during surgery). More typically, non-surgical and/or non-invasive administration is used. For instance, a conventional controllable endoscopic delivery device can be used so long as the needle lumen or bore is of sufficient diameter (e.g. 30 gauge or larger) that shearforces will not damage the cells being delivered. The in vitro pre-conditioned PMSCs may be administered in a manner that permits them to graft to the intended target site and induce the regeneration of cartilage tissue in the functionally deficient area.

In some embodiments, the target site to which the PMSCs are administered is in a joint.

In some embodiments the in vitro pre-conditioned PMSCs migrate after administration to a target site.

The subject to which the in vitro pre-conditioned PMSCs are administered may be a mammal, including human and non-human primates, domestic animals and livestock, pet or sports animals, for example, dogs, horses, cats, sheep, pigs, and cows. Typically, however, the subject is a human subject. The in vitro pre-conditioned PMSCs may be from the subject's own body (autologous transplant) or from a donor (allogeneic transplant).

Kits

Any of the pharmaceutical compositions of the invention described herein can be used together with a set of instructions, i.e., to form a kit. The kit may include instructions for use of the pharmaceutical compositions as a therapy as described herein. For example, the instructions may provide dosing and therapeutic regimes for use of the compounds of the invention to reduce symptoms and/or underlying cause of the OA or pre-OA.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I claim:

1. A composition comprising:
an isolated in vitro pre-conditioned population of placenta derived mesenchymal stem cells (PMSCs),
wherein the PMSCs promote de novo regeneration of cartilage when the PMSCs are administered to a subject.

2. The composition of claim 1, wherein the in vitro pre-conditioning comprises providing a population of mesenchymal stem cells from a placenta, and culturing the population of mesenchymal stem cells in a medium comprising soluble Notch ligand Jagged1 (JAG1).

3. The composition of claim 2 wherein the JAG1 is in a concentration of between 5.0 μg/ml and 15 μg/ml.

4. The composition of claim 2 wherein the JAG1 is in a concentration of substantially 10 μg/ml.

5. The composition of claim 1 wherein the PMSCs are harvested from a villous tissue of the placenta.

6. A method of treating one of osteoarthritis, cartilage damage or loss, and pre-osteoarthritis in a patient comprising:
administering to the patient a pharmaceutical composition comprising
an isolated in vitro pre-conditioned population of placenta derived mesenchymal stem cells (PMSCs), wherein the in vitro pre-conditioning comprises providing a population of mesenchymal stem cells from a placenta, and culturing the population of mesenchymal stem cells in a medium comprising soluble Notch ligand Jagged1 (JAG1); and
a pharmaceutically acceptable carrier.

7. The method of claim 6 wherein the pharmaceutical composition is injected into a joint of the patient.

8. The method of claim 7 wherein the joint is a knee.

9. The method of claim 6 wherein the pharmaceutical composition contains between 0.1 million and 300 million PMSCs.

10. The method of claim 6 wherein the pharmaceutical composition contains between 0.2 million and 30 million PMSCs.

11. The method of claim 6 wherein the pharmaceutical composition contains between 0.5 million and 3 million PMSCs.

12. The method of claim 6 further comprising making no more than one injection of the pharmaceutical composition into the patient a week.

13. The method of claim 12 further comprising making one injection of the pharmaceutical composition into the patient a week for four weeks.

14. The method of claim 6 wherein the pharmaceutical composition contains JAG1.

15. The method of claim 14 wherein JAG1 is in a concentration of between 5.0 μg/ml and 15 μg/ml.

16. The method of claim 14 wherein the JAG1 is in a concentration of substantially 10 μg/ml.

17. The method of claim 6 wherein the PMSCs are harvested from a villous tissue of the placenta.

18. A pharmaceutical composition comprising:
placenta derived mesenchymal stem cells (PMSCs),
Notch ligand Jagged1 (JAG1); and
a pharmaceutically acceptable carrier.

19. The pharmaceutical composition of claim 18 wherein the wherein the PMSCs are harvested from a villous tissue of a human placenta.

20. The pharmaceutical composition of claim 19 wherein the JAG1 is in a concentration of between 8.0 μg/ml and 12.0 μg/ml.
